(19) United States
(12) Patent Application Publication
(10) Pub. No.: US 2010/0234812 A1
(43) Pub. Date: Sep. 16, 2010

(54) Fluid Recovery System

(75) Inventors: Lance T. Parker, Houston, TX (US); Kevin J. Derichs, Buda, TX (US)

Correspondence Address: JONES, WALKER, WAECHTER, POITEVENT, CARRERE;& DENEGRE, L.L.P., 5TH FLOOR, FOUR UNITED PLAZA, 8555 UNITED PLAZA BOULEVARD, BATON ROUGE, LA, 70809, US

(73) Assignee: ANIMAL INNOVATIONS, INC., Houston, TX (US)

(21) Appl. No.: 12/683,523

(22) Filed: Jan. 7, 2010

Related U.S. Application Data
(60) Provisional application No. 61/209,755, filed on Mar. 11, 2009.

(51) Int. Cl. A61M 5/31

(52) U.S. Cl. 604/218

(57) ABSTRACT

With the use of a selectable fluid path assembly, the recovery and cleaning of the liquid path line is possible. Selecting the primary use path permits a syringe assembly to be filled with fluid in a positive pressure supplied fluid system. When the secondary fluid path is selected, the fluid in the line can be reversed and with negative pressure pulled back through the line up to the pump motor and moved back into the bottle. With air following the liquid a bubble can be detected and stop the fluid reversal. With the use of a selectable fluid path assembly, the prevention of contamination of the remaining liquid in the bottle can be prevent through proper use of the fluid recovery system.

### PRIORITY CLAIM

This application claims the priority benefit of U.S. provisional application No. 61/209,755 filed on Mar. 11, 2009, the contents of which are hereby incorporated by reference.

### FIELD OF INVENTION

This invention relates to syringe injection systems and methods of recovery of fluids from syringe injection systems.

### BACKGROUND OF THE INVENTION

It is often desirable to treat large numbers of individuals or animals with a substance, such as a medication or other material, with speed, efficiency, accuracy, and accurate maintenance of records. As an example, the livestock industry requires routine vaccinating, medicating and/or treating of cattle or livestock. Failure to properly treat the animals can result in significant losses to the rancher or feedlot owner or other party responsible for the livestock. Typically, the livestock is segregated into groups according to general size and weight. It is common upon arrival at the processing station for cattle to be vaccinated for viral respiratory disease, implanted with a growth stimulant, and treated for internal and external parasites. In high stress situations, antibiotics are sometimes administered simultaneously with vaccinations.

To assist in vaccination large numbers of animals, able syringe injections systems have been developed that allow a syringe to be filled by a pump from a fill bottle, where the dose loaded into the syringe can be effectively controlled and varied as needed to tailor the injections by animal weight. Such a syringe system does not require the cumbersome filling of the syringe from a separate fluid container, allows for repeated injections, using precisely predetermined but differing dosages, and are capable of operating in a wide range of environments. One such syringe system is shown in U.S. Pat. No. 7,056,307, hereby incorporated by reference As shown in FIG. 1, a syringe system will include a fill or reservoir bottle 2, a syringe 10, a highly accurate reversible motor 4 and pump 4 combination, and various fluid lines between the components. The system unit pump 4 is a valveless, substantially viscosity-independent pump. The pump 4 used in the system is manufactured by Fluid Metering, Inc. (“FMI”) of Syosset, N.Y., Models STH and STQ. To the extent necessary to understand the features and construction of the pump 4 manufactured by FMI, Applicant hereby incorporates by reference U.S. Pat. Nos. 5,279,210; 5,246,354; 5,044,889; 5,020,980; 5,015,157; and 4,941,809. A complete FMI pump cycle includes a ½ cycle of gather fluid from the reservoir lines, and a second ½ cycle of pumping the gathered fluid out the fluid line 6 (that is, the pump is not continuously pumping fluids as would, for instance, and impeller type pump). However, instead of use of a reversible motor/pump, two pumps may be used, (one pumping to the syringe, one pumping from the syringe), and a switch employed to select the desired pump.

Animal medicine injection dispensing systems used expensive medications which are wasted when the remaining amount left in the line is not used for medicating. Often, the fluid bottle is removed from the system, and materials remaining in the fluid lines and syringe are wasted, disposed of by dispensing the materials out through the dispensing tip, or in some cases, dispense back into the bottle for later use. This is poor practice, as the dispensing tip could be contaminated and returning materials through the tip could contaminate the remaining volume in the bottle. A system is needed to easily recover fluids in a syringe system without the possibility of contamination.

### SUMMARY OF THE INVENTION

The fluid recovery system includes two fluid paths, a first path that preferably terminates in a check valve or other one way valve, though it could open to directly to the atmosphere, and a second part that terminates into the syringe, such as at a syringe valve body. The system also includes a switch that allows the fluid line from the pump to be switched between the two paths.

### BRIEF DESCRIPTION OF THE DRAWINGS

FIG. 1 is a block diagram of representation of a basic syringe system.

FIG. 2 is one embodiment of a syringe that may be used in the syringe system.

FIG. 3a is a cross section side view of a plunger embodiment of the fluid recovery system in direct path mode.

FIG. 3b is a cross section side view of a plunger embodiment of the fluid recovery system in vent path mode.

FIG. 4a is a detail cross section side view of a first plunger embodiment of the fluid recovery system in direct path mode.

FIG. 4b is a detail cross section side view of a first plunger embodiment of the fluid recovery system in vent path mode.

FIG. 5a is a cross section side view of a second plunger embodiment of the fluid recovery system in direct path mode.

FIG. 5b is a cross section side view of a second plunger embodiment of the fluid recovery system in vent path mode.

FIG. 6a is a detail cross section side view of a rotatable embodiment of the fluid recovery system in direct path mode.

FIG. 6b is a detail cross section side view of a rotatable embodiment of the fluid recovery system in vent path mode.

FIG. 7a is a cross section side view of a rotatable embodiment of the fluid recovery system in direct path mode.

FIG. 7b is a cross section side view of a rotatable embodiment of the fluid recovery system in vent path mode.

### DETAILED DESCRIPTION OF THE INVENTION

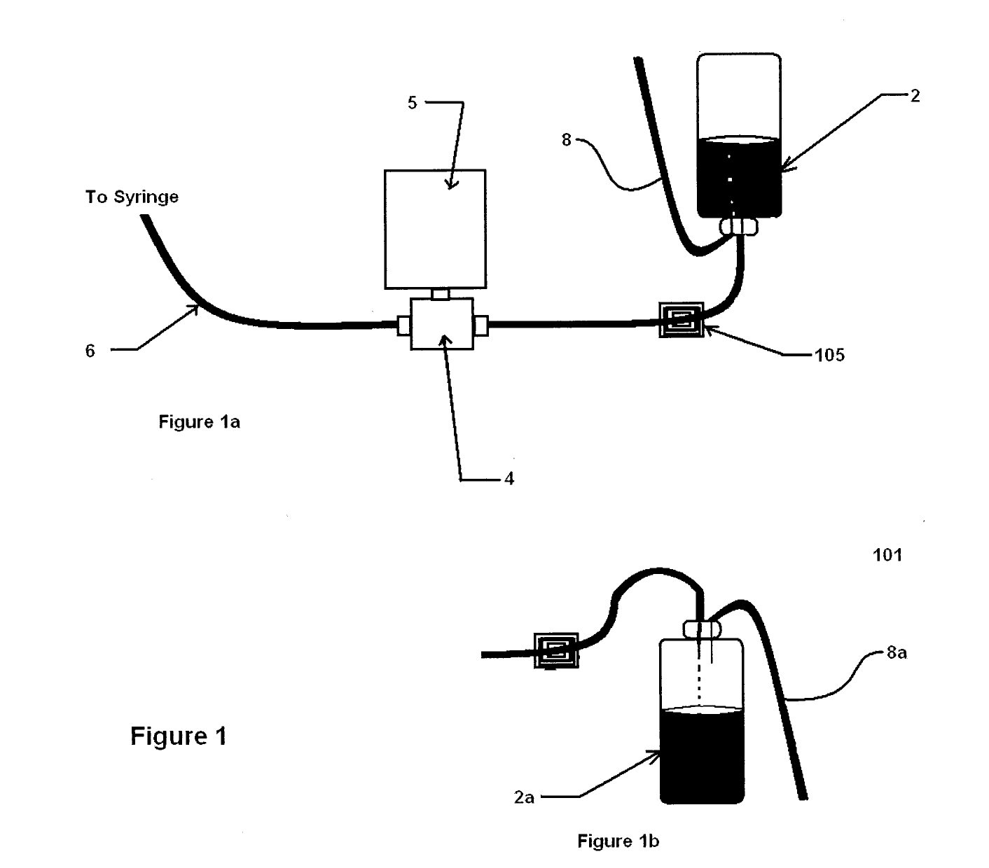

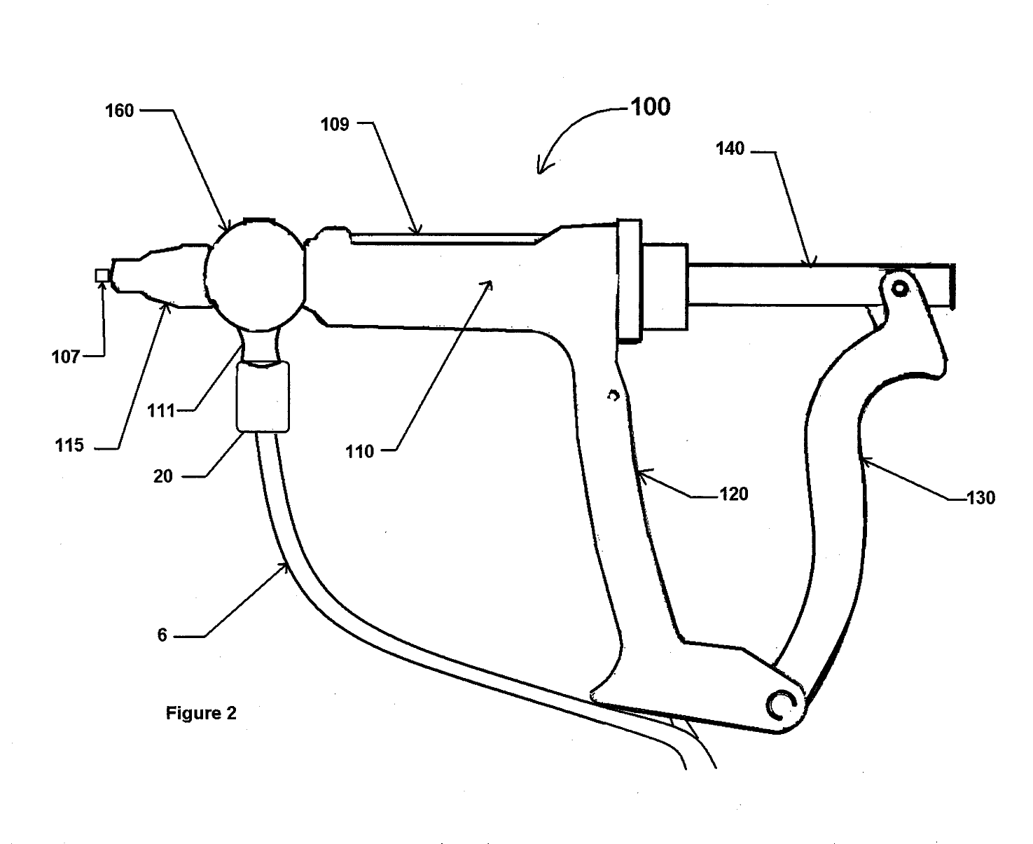

The invention will be described within a syringe system, where a syringed is filled by operation of a reversible pump, such as described in U.S. Pat. No. 7,056,307 or 6,989,000, both of which are incorporated by reference. A basic system is shown in FIG. 1. As shown in FIG. 1, a syringe system will include a fill or reservoir bottle 2, a pump 4 and motor 5, various fluid lines between the components and a syringe 100 shown in FIG. 2. Fluid is pumped from the reservoir bottle 2 to the syringe 100 (shown in FIG. 2) and enters the syringe at a syringe inlet 111 which controls the flow of fluids within the syringe 100.

A schematic of one syringe embodiment is shown in FIG. 2. As shown, the syringe 100 is a includes a syringe body 110 having a front grip 120 and rear grip 130, with one of the grips movable with respect to the other (here the rear grip 130 is movable with respect to an integral front grip 120). The two gripped syringe is preferred, but not required (e.g., the plunger could be separately operated). The syringe 100 includes a dispensing tip 115 to which a needle may be attached. The syringe body 110 includes a hollow barrel chamber 109 with a shaft plunger 140 slidable in the interior of barrel chamber 109. By squeezing the two grips, the shaft plunger 140 is forced into the barrel chamber 109 to discharge fluids stored in the barrel chamber 109 through the dispensing tip 107, and ultimately, to an injection needle (not shown) attached to the dispensing tip 107. Most pump finable syringes include a valve body 160 having internal valves to control fluid movement within the syringe 100, such as the valve bodies depicted in U.S. Pat. No. 6,989,000. For purposes of the fluid recovery system, there are not preferred syringe valve systems. Fluids enter the syringe through the syringe inlet 111, here shown as being on the valve body 160.

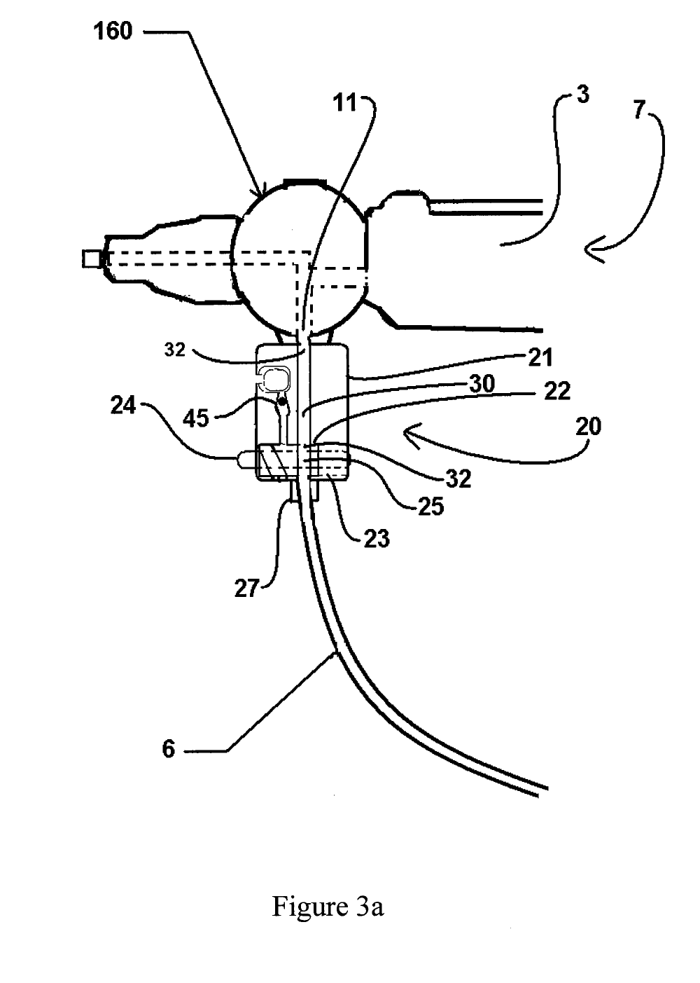

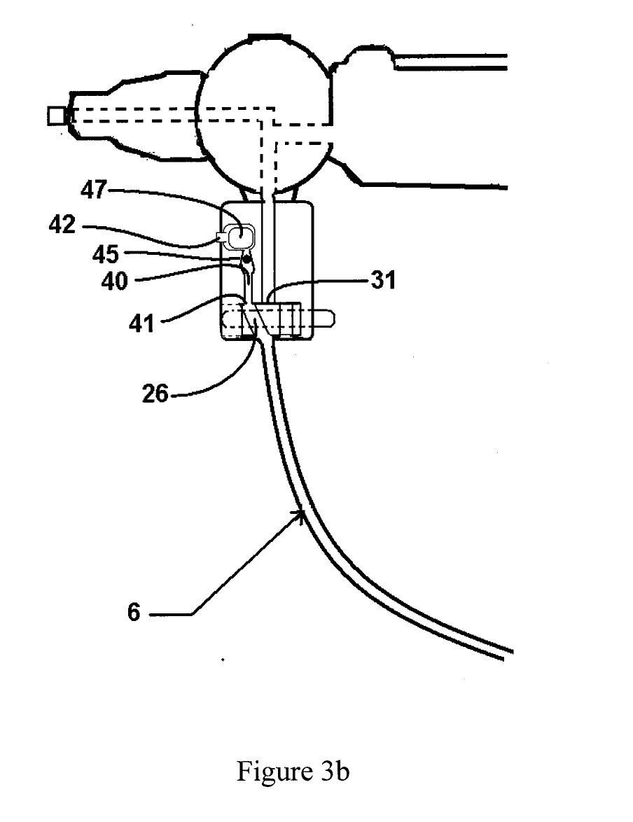

To this base syringe injection system is added a fluid recovery system 20. In the embodiment shown in FIG. 2, the recovery system is located at the input of the fluid line 6 to the syringe 100 (that is, at the syringe inlet). As shown, the recovery system is shown attached to a syringe valve body 160, however, the fluid recovery system 20 could be placed anywhere on the fluid line 6. It is preferred to attach the recovery system directly to the syringe 100, to provide for the efficient recovery of fluids, later described. In the embodiment shown in FIGS. 3a & 3b, the system includes a path body 21 and a fluid switch 22. Path body 21 is a block (such as inert plastic, stainless, aluminum) having two channels or fluid paths therethrough: a direct path 30, and a vent path 40. Both paths have an inlet 41 and 31 respectfully, and outlets 42 and 32 respectfully. Vent path 40 contains in a check valve 45 or other means to removably seal vent path (such as an insertable plug, threaded cover, etc). As shown, the check valve 45 is positioned near the vent path outlet 42. Vent path may simply open to the atmosphere, but this is not preferred. An air filter 47 may also be positioned in the vent path 40, or at the vent path outlet 42. Also a cap or cover is preferably used to cover the terminal end of the vent path when not in use.

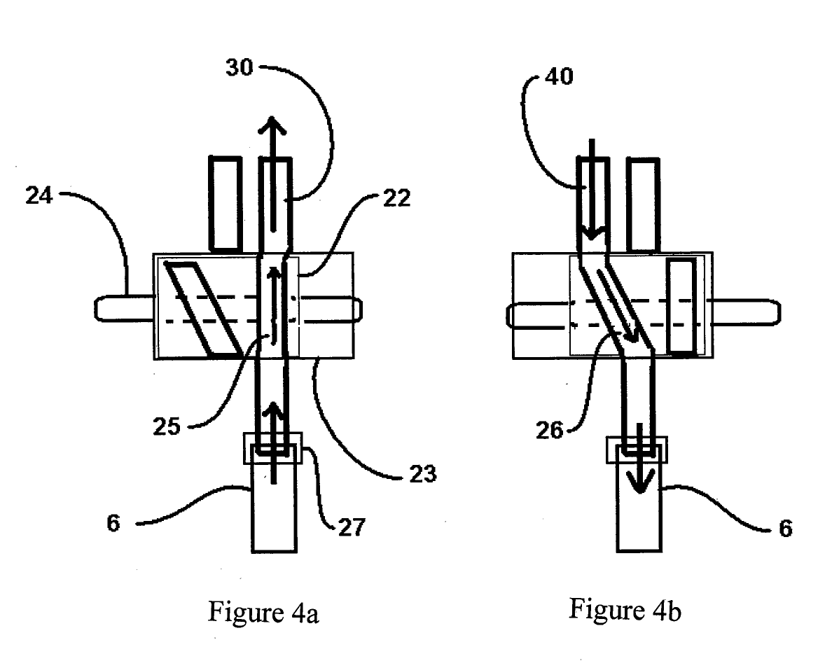

As shown in FIGS. 4a & 4b, fluid switch 22 is a housing 23 having a slidable plunger 24 positioned therein. Fluid line 6 is coupled to the switch housing 23 (such as with a quick connect joint) at connect 27. Two hollow tubes or channels are fixedly positioned on the plunger 24: a direct switch channel 25 and a vent switch channel 26, each having inlets and outlets (the paths may be channels drilled through the plunger, or tubes attached to the plunger) By sliding the plunger 24 between a first position (shown in FIGS. 3a & 4a) to a second position (shown in FIGS. 3b & 4b), the operator can choose to connect either the direct switch channel 25, or vent switch channel 26, to the fluid line 6.

In the plunger position shown in FIG. 3a, the switch position fluidly connects direct switch channel 25 between fluid line 6 and direct path 30, while blocking the vent path inlet 41. In this position, the direct path outlet 32 is sealingly aligned with the syringe inlet 11 (here shown on the valve body 160), and direct path inlet is sealingly aligned with direct switch channel 25. In the plunger position shown in FIG. 3b, vent switch channel 26 is fluidly connected between fluid line 6 and the vent path 40, while the direct path inlet 31 is blocked, thereby connecting fluid line 6, through the vent path 40 and check valve 45 (if so equipped), to the atmosphere.

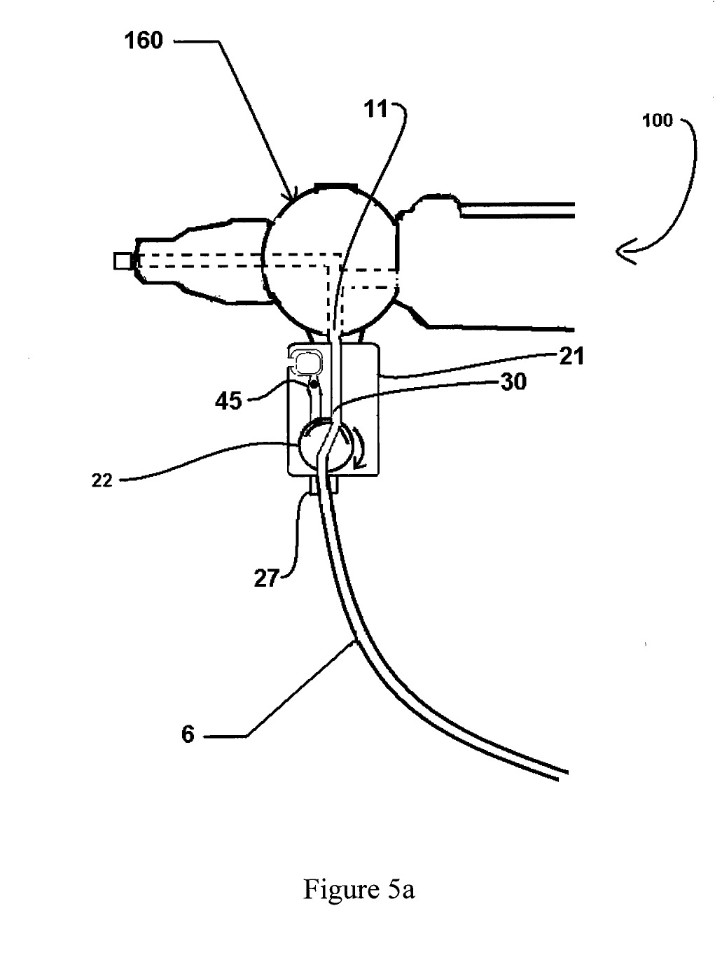

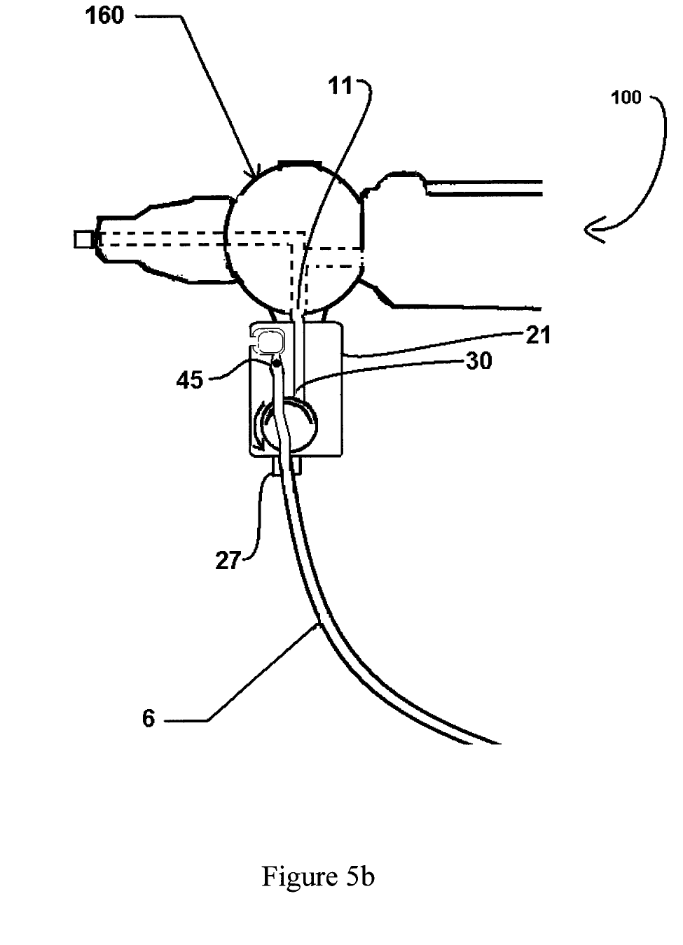

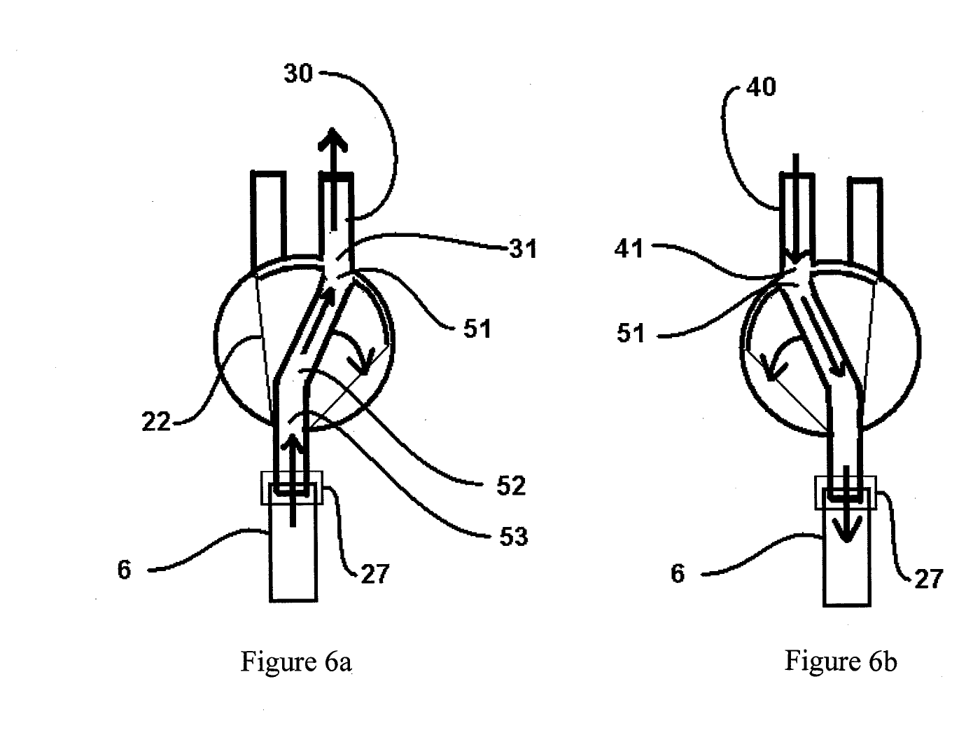

In a second embodiment shown in FIGS. 5a & 5b, the recovery system is located at the input of the fluid line 6 to the syringe 100 (that is, at the syringe inlet). As shown, the recovery system is shown attached to a syringe valve body 160. This embodiment uses a type of fluid switch 22 that is a rotatable fluid switch. One embodiment of a rotatable fluid switch is shown in FIGS. 5a & 5b. In this embodiment, the switch body 22 is rotatably attached within the path body 21. Rotation may be in a vertical plane (e.g. in a plane parallel to the plane containing syringe handles), or a horizontal plane (e.g. in a plane perpendicular to the plane containing syringe handles) or even an intermediary plane. If rotation occurs in a vertical plane, as shown in FIGS. 5a, 5b, 6a and 6b, it is preferred that the lower surface of the path body 21 and the upper surface of the fluid switch housing 22 be curved to accommodate a seal between the two bodies (see detail of FIG. 5a).

Within the rotatable fluid switch housing 22 is a single fluid path or fluid channel 52. The fluid switch housing 22 is rotatable between two positions, a first position connecting the single path outlet 51 to the direct path inlet 31 (see FIG. 5a), and a second position, connecting the single path outlet 51 to the vent path inlet 41 (see FIG. 5b). The fluid line 6 remains connected to the single path inlet 53, and hence moves with the rotation of the fluid switch housing 22.

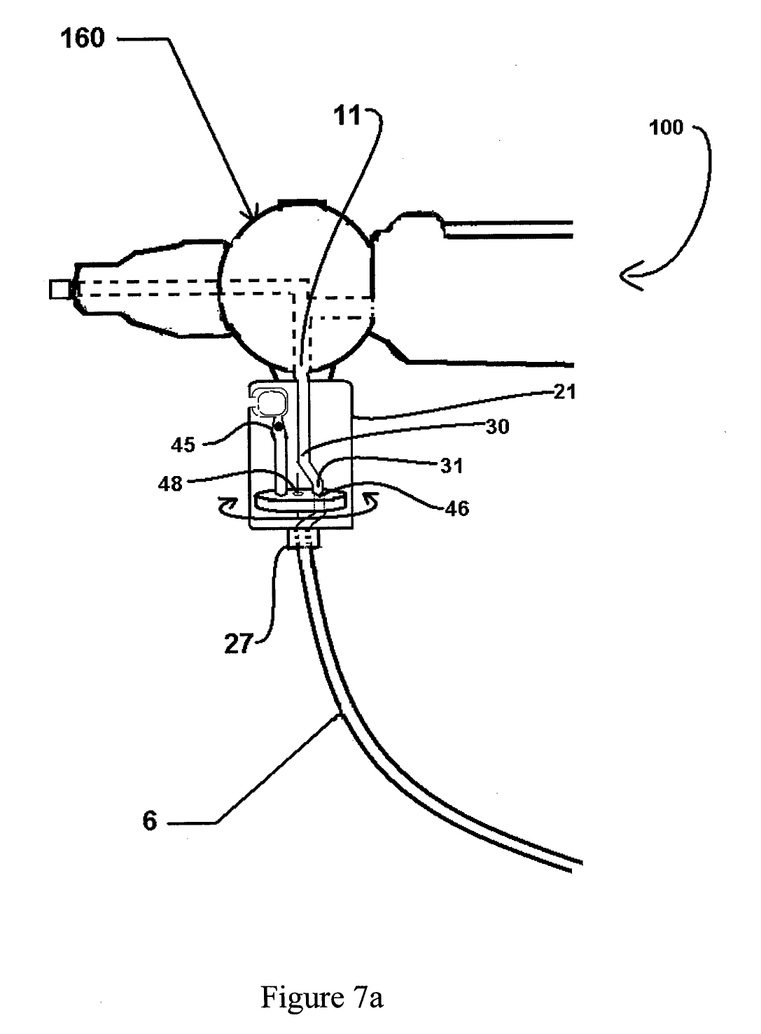

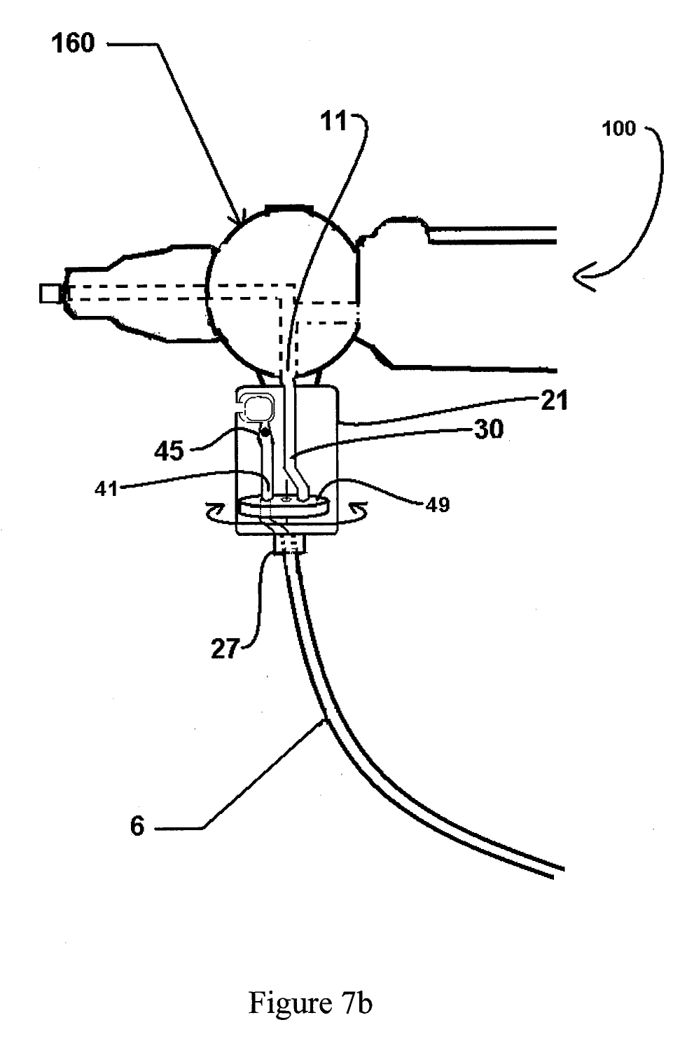

If rotation occurs in a horizontal plane, as shown in FIGS. 7a & 7b, a rotatable disk 49, rotating around a pivot point 48, is rotatable between two positions, a first position connecting a single disk fluid channel outlet 46 to the direct path inlet 31 (see FIG. 7a), and a second position, connecting the single disk fluid channel outlet 46 to the vent path inlet 41 (see FIG. 7b). The fluid line 6 remains connected to the single disk fluid channel outlet 46, and hence moves with the rotation of the rotatable disk 49.

Other embodiments of a plunger type switch are may also be utilized. For instance, the plunger may carry a single tube 50, where the tube inlet remains connected to the fluid line 6, and the tube outlet is movable by operation of the plunger between the vent path 40 and direct path 30. In this configuration, that portion of the tube 50 near the tube inlet must bend or flex between the two positions, and for this reason, is not preferred—the flexing or bending of the tube 50 creates a potential fracture point in the flexible tube. An alternative arrangement using a single channel 50 in the switch housing that avoids the need for flexing can be achieved by allowing the switch housing 22 to be slidable with respect to the path body 21. In this embodiment (not shown), the input fluid line 6 remains connected to the inlet of the channel 50, and the switch housing 22 acts as a plunger, slidable between a first position (connecting the single path 50 outlet to the vent path inlet 41 and blocking the direct path inlet), and a second position (connecting the single path 50 outlet to the direct path inlet 31, and blocking the vent path inlet).

### Fluid Recovery Operation

The fluid recovery system thus has two operator selected operating positions: a first fill position, fluidly connecting the fluid line 6, through the recovery system (direct path), to the inlet of the syringe, and a second recovery position that fluidly connects the fluid line 6, through the recovery system (vent path), to a vent and the atmosphere. When the fill position is selected, the pump 4 and motor 5 should be placed in the normal mode, pumping fluids from the reservoir bottle 2 through the pump 4 to the flow control system, and then to the syringe. When the recovery position is selected, the motor 5 pump 4 should be in reverse mode, pumping fluids from the fluid line 6, through the pump 4, back to an up-righted reservoir bottle 2a. In the recovery position, the fluid line 6 is connected to the atmosphere via the vent path 40 in the fluid recovery system, so no back pressure will be created when pumping from the syringe.

In all full fluid line recovery operations, it is preferred that reservoir bottle 2a be up-righted (See FIG. 1b) to prevent the build up of pressure in the reservoir bottle 2a, which could result in fluid being pushed out the bottle vent. Additionally, shown in the fluid line to the reservoir bottle 2 is a bubble detector 105, such as a Lifeguard ultrasonic air bubble detector from MOOG, Inc. of Stuttgart, Germany).

During partial fluid line recovery operations, which can occur during the syringe fill operation, fluid is extracted from the reservoir bottle 2 by the motor 5 pump 4, as the fluid level goes down, air will be pulled into the fluid line where the bubble detector 105 can be tied to a motor control to send a command to shut off the motor and prompt the operator (to replace the empty bottle with a replacement bottle) when bubbles are detected in the fluid line. After a fluid bottle is replaced the motor can be controlled to run in reverse to push the air out of the line between the sensor and the reservoir bottle 2, then back to forward to pull liquid into the line replacing the air, this will insure that there will be no air in the fluid line that could end up in the syringe, causing the incorrect volume to be dispensed.

1. An improved pump operated syringe, comprising a syringe having a barrel chamber for storing medications, a dispensing tip adapted for holding a needle, said dispensing tip fluidly connectable to said barrel chamber, a syringe inlet fluidly connectable to said barrel chamber, a plunger shaft slidable in said barrel chamber, wherein the improvement comprises
a fluid recovery system, said fluid recovery system comprising an path body and a switch housing, said path body comprising a direct path and a vent path, said vent path having a vent inlet and a vent outlet and a vent path fluid channel therebetween, said direct path having a direct path inlet and a direct path outlet and a direct path channel therebetween, said direct path outlet fluidly connected to said syringe inlet,
said switch housing having a switch inlet and a switch outlet, and a switch path channel therebetween, said path switch channel movable to a fill position and a recovery position, where said fill position comprises said direct path inlet being fluidly connected to said switch path outlet, and where said recovery position comprises said switch path outlet being fluidly connected to said vent path inlet and said direct path inlet is blocked.

2. The improved syringe system of claim 1 wherein said path switch channel is slidable with respect to said path body.

3. The improved syringe system of claim 1 wherein said path switch channel is rotatable with respect to said path body.

4. The improved syringe system of claim 1 wherein said vent path further comprises a sealable vent,

5. The improved syringe system of claim 4 wherein said sealable vent comprises a check valve.

6. An improved pump operated syringe, comprising a syringe having a barrel chamber for storing medications, a dispensing tip adapted for holding a needle, said dispensing tip fluidly connectable to said barrel chamber, a syringe inlet fluidly connectable to said barrel chamber, a plunger shaft slidable in said barrel chamber, wherein the improvement comprises
a fluid recovery system, said fluid recovery system comprising an path body and a switch housing, said path body comprising a direct path and a vent path, said vent path having a vent inlet and a vent outlet and a vent path fluid channel therebetween, said direct path having a direct path inlet and a direct path outlet and a direct path channel therebetween, said direct path outlet fluidly connected to said syringe inlet,
said switch housing having a direct switch path having an inlet and an outlet and a direct switch channel therebetween, and a vent switch path having an inlet and an outlet and a vent switch path therebetween,
said fluid recovery system adapted to switch between a fill position and a recovery position, where said fill position comprises said direct path inlet being connected to said switch path outlet, and where said recovery position comprises said direct path inlet being closed and said vent path inlet being fluidly connected to said switch path outlet.
